(19) United States
(12) Patent Application Publication
(10) Pub. No.: US 2026/0243665 A1
(43) Pub. Date: Aug. 20, 2026

(54) SOIL SAMPLING SYSTEM

(71) Applicant: The Regents of the University of California, Oakland, CA (US)

(72) Inventors: Cassandra Bonfil, Oakland, CA (US); Isaya Kisekka, Oakland, CA (US)

(21) Appl. No.: 19/544,581

(22) Filed: Feb. 19, 2026

Related U.S. Application Data
(60) Provisional application No. 63/760,215, filed on Feb. 19, 2025.

(51) Int. Cl. G01N 21/33; G01N 1/14; G01N 33/24

(52) U.S. Cl. CPC G01N 21/33; G01N 1/14; G01N 33/246

(57) ABSTRACT

An example soil nitrate sensing system includes: a suction lysimeter; a soil moisture sensor; a spectrometer; and a controller, wherein the controller is configured to: estimate a soil moisture measurement by the soil moisture sensor; based on the soil moisture measurement, acquire a water sample by the suction lysimeter; and estimate nitrate concentrations as NO₃—N in the soil porewater sample using the spectrometer.

### CROSS-REFERENCE TO RELATED APPLICATIONS

This application claims the benefit of U.S. provisional patent application No. 63/760,215, filed on Feb. 19, 2025, and titled “SOIL SAMPLING SYSTEM,” the disclosure of which is expressly incorporated herein by reference in its entirety.

### BACKGROUND

Soil lysimeters are systems for measuring water and solutes in soil. A lysimeter collects water from the soil for analysis. Lysimeters generally include devices configured to trap water and one or more sensors to measure the trapped water. Lysimeters can be important for measuring pollution (e.g., fertilizer leaching) and/or studying any other parameter of the soil. Improvements to lysimeters can improve agriculture and science by improving fertilizer management.

### SUMMARY

In some aspects, implementations of the present disclosure include a soil nitrate sensing system including: a suction lysimeter; a soil moisture sensor; a spectrometer; and a controller, wherein the controller is configured to: monitor a soil moisture measurement by the soil moisture sensor; based on the soil moisture measurement, acquire a water sample by the suction lysimeter; and estimate nitrate concentrations as NO3-N in the water sample using the spectrometer.

In some aspects, implementations of the present disclosure include a soil nitrate sensing system, wherein the soil moisture sensor includes a volumetric water content sensor.

In some aspects, implementations of the present disclosure include a soil nitrate sensing system, wherein estimating a soil moisture measurement includes estimating a soil matric potential.

In some aspects, implementations of the present disclosure include a soil nitrate sensing system, wherein the soil matric potential is computed by a Van Genuchten model.

In some aspects, implementations of the present disclosure include a soil nitrate sensing system, wherein the suction lysimeter includes at least four suction probes.

In some aspects, implementations of the present disclosure include a soil nitrate sensing system, wherein the suction lysimeter is configured to sample water from at least two or four locations.

In some aspects, implementations of the present disclosure include a soil nitrate sensing system, wherein the soil moisture sensor includes at least two sensor probes.

In some aspects, implementations of the present disclosure include a soil nitrate sensing system, wherein the soil moisture measurement includes soil moisture at two locations.

In some aspects, implementations of the present disclosure include a soil nitrate sensing system, wherein the spectrometer includes a UV-Vis spectrometer.

In some aspects, implementations of the present disclosure include a soil nitrate sensing system, wherein the spectrometer is configured to measure light between 200 and 750 nanometers.

In some aspects, implementations of the present disclosure include a soil nitrate sensing system, wherein estimating a nitrate (NO3-N) concentration includes estimating a compensated spectrum.

In some aspects, implementations of the present disclosure include a soil nitrate sensing system, wherein the soil lysimeter includes a vacuum pump, a peristaltic pump, and a plurality of Duran bottles configured in the controller to store a plurality of soil porewater samples based on sampling sequence.

In some aspects, implementations of the present disclosure include a soil nitrate sensing system, wherein the soil lysimeter is configured to vent the plurality of bottles.

In some aspects, implementations of the present disclosure include a computer-implemented method of nitrate (NO3-N) measurement including: initiating a soil moisture measurement; acquiring a soil porewater sample by applying a vacuum prior to application of a force on a suction lysimeter based on the soil moisture measurement; and estimating a nitrate concentration of the soil porewater sample using a spectrometer.

In some aspects, implementations of the present disclosure include a computer-implemented method, wherein estimating a soil moisture measurement includes estimating a soil matric potential.

In some aspects, implementations of the present disclosure include a computer-implemented method, wherein the soil matric potential is between −30 kPa and −1500 kPa.

In some aspects, implementations of the present disclosure include a computer-implemented method, wherein the soil matric potential is computed by a Van Genuchten model.

In some aspects, implementations of the present disclosure include a computer-implemented method, wherein the Van Genuchten model includes: 20.

In some aspects, implementations of the present disclosure include a computer-implemented method, wherein estimating a nitrate concentration of the soil porewater sample using a spectrometer includes estimating a compensated spectrum which minimizes effects of Dissolved Organic Carbon on nitrate measurements.

In some aspects, implementations of the present disclosure include a computer-implemented method, further including repeating steps of acquiring a second water sample and estimating an additional nitrate concentration of the second water sample.

In some aspects, implementations of the present disclosure include a computer-implemented method, further including transmitting the nitrate concentration to a remote computing device.

In some aspects, implementations of the present disclosure include a computer-implemented method, further including determining an amount of nitrate to apply to a field based on the nitrate concentration.

In some aspects, implementations of the present disclosure include a computer-implemented method, further including applying a nitrate fertilizer to a field based on the nitrate concentration.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A illustrates a block diagram of a lysimeter, according to implementations of the present disclosure.

FIG. 1B illustrates a block diagram of a lysimeter, according to implementations of the present disclosure.

FIG. 2A illustrates an example method of operating a lysimeter, according to implementations of the present disclosure.

FIG. 2B illustrates an example method of operating a lysimeter, according to implementations of the present disclosure.

FIG. 2C illustrates an example method of operating a lysimeter, according to implementations of the present disclosure.

FIG. 3 illustrates an example computing device.

FIG. 4 illustrates an example implementation of the present disclosure deployed in a tomato field.

FIG. 5 illustrates a perspective view of the interior of the example implementation shown in FIG. 4.

FIG. 6 illustrates a rear-view of the example implementation of the present disclosure shown in FIG. 4.

FIG. 7 illustrates a rear-view of the example implementation of the present disclosure shown in FIG. 4.

FIG. 8 illustrates an interior view of the example implementation of the present disclosure shown in FIG. 4.

FIG. 9 illustrates an interior view of the example implementation of the present disclosure shown in FIG. 4.

FIG. 10 illustrates an interior view of the example implementation of the present disclosure shown in FIG. 4.

FIG. 11 illustrates an interior view of the example implementation of the present disclosure shown in FIG. 4.

FIG. 12 illustrates a rear-view of the example implementation of the present disclosure shown in FIG. 4.

FIG. 13 illustrates a rear-view of the example implementation of the present disclosure shown in FIG. 4.

FIG. 14 illustrates an example user interface configured to output data collected from the example implementation shown in FIGS. 4-13, the example data shown represents an example deployment and testing period for the example implementation of the present disclosure.

FIG. 15 illustrates an example user interface configured to display soil moisture (as SMT 02 humidity) and nitrate sensor data (as NO3), according to the example implementation shown in FIGS. 4-13.

FIG. 16 illustrates example user interfaces configured to display soil nitrate concentrations, according to the example implementation shown in FIGS. 4-13.

FIG. 17 illustrates a display of an example nitrate measurement event, according to an example implementation of the present disclosure.

FIG. 18 illustrates results comparing an example implementation of the present disclosure to a conventional suction lysimeter, according to a study of an example implementation of the present disclosure.

FIG. 19 illustrates results showing the nitrate-N concentrations at multiple depths (e.g., 30, 45, and 60 cm) and lateral positions within the variably saturated zone surrounding an SDI emitter, capturing dynamic nitrate responses to discrete fertigation events and subsequent redistribution over time, according to a study of an example implementation of the present disclosure.

FIG. 20 illustrates volumetric water content measured by an example implementation of the present disclosure.

FIG. 21 illustrates matic potential measured by an example implementation of the present disclosure.

FIG. 22 illustrates an example system layout for an example implementation of the present disclosure.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for measuring nitrates, it will become evident to those skilled in the art that the implementations are not limited thereto, but are applicable for any type of lysimetry or lysimeter.

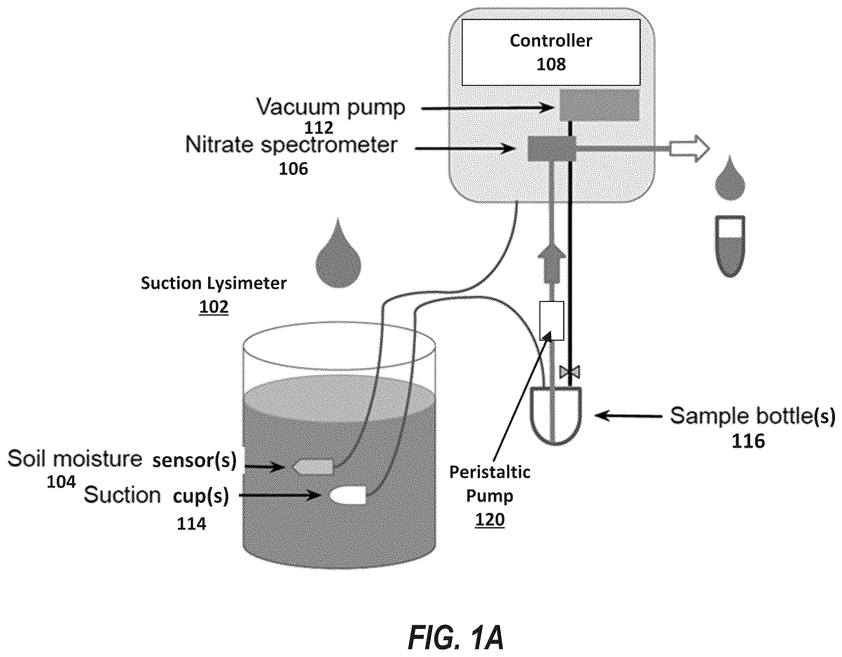

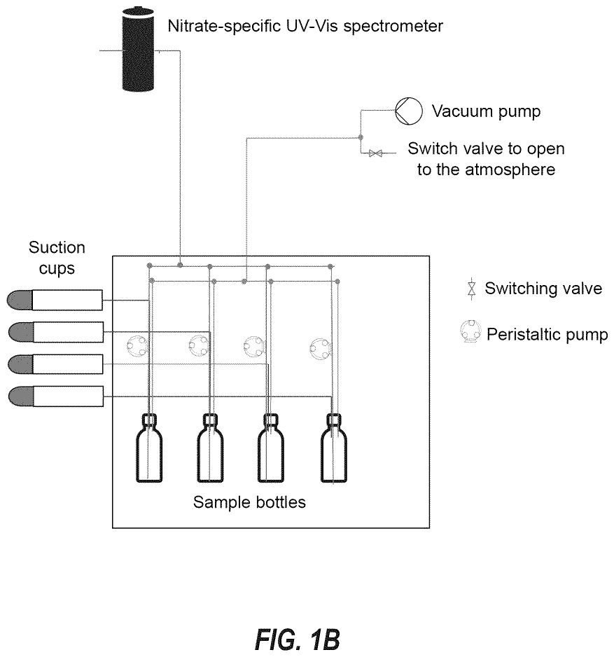

An example soil nitrate sensing system according to the present disclosure is shown in FIG. 1A. The soil nitrate sensing system includes a suction lysimeter 102, a soil moisture sensor 104, a spectrometer 106, and a controller 108.

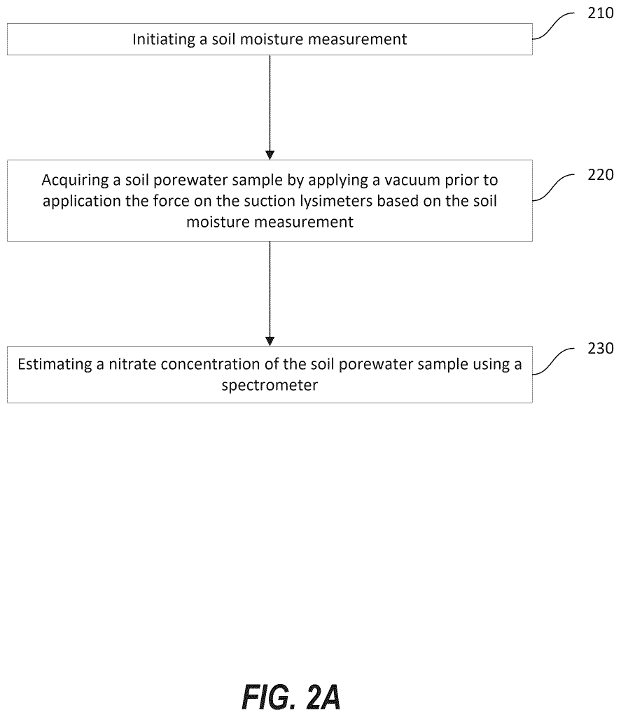

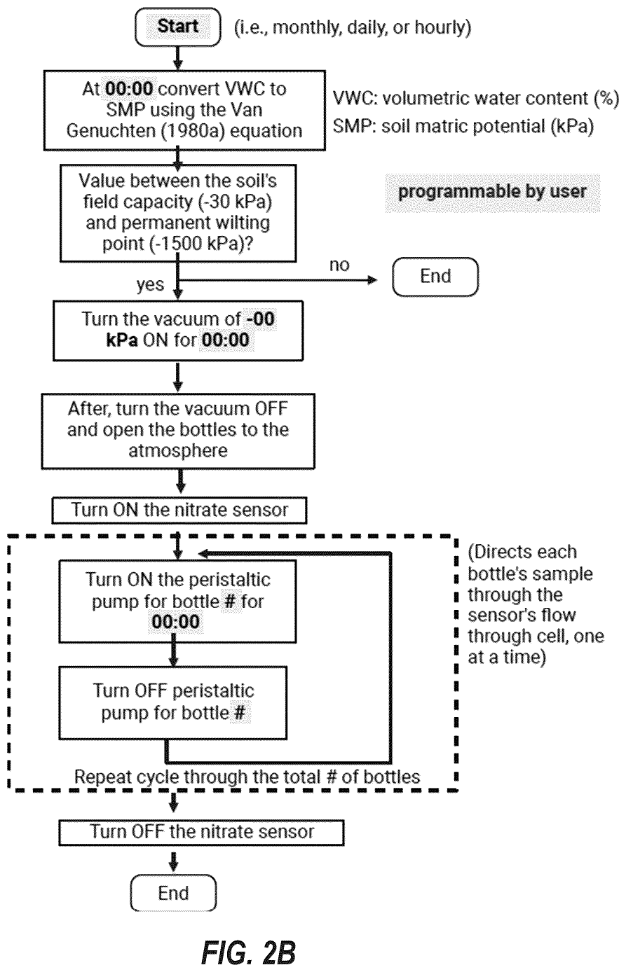

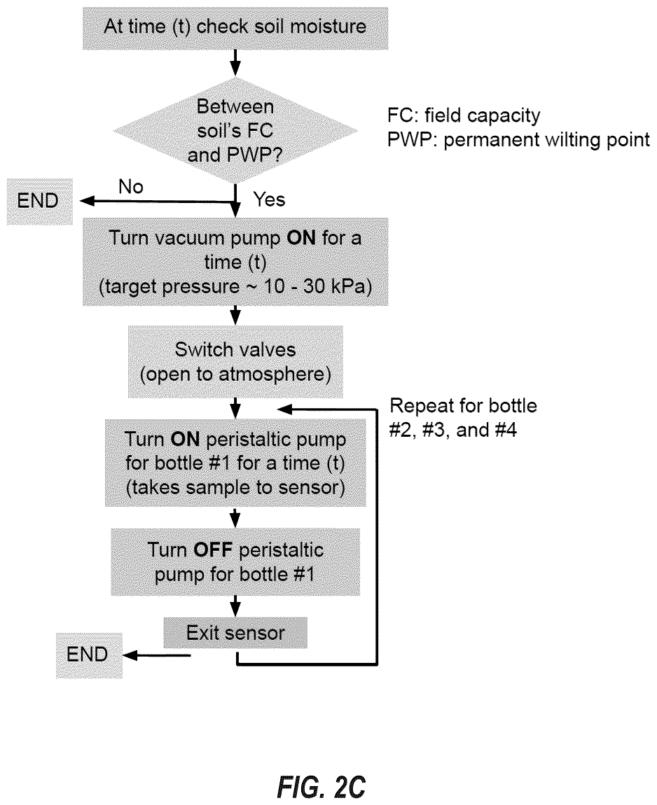

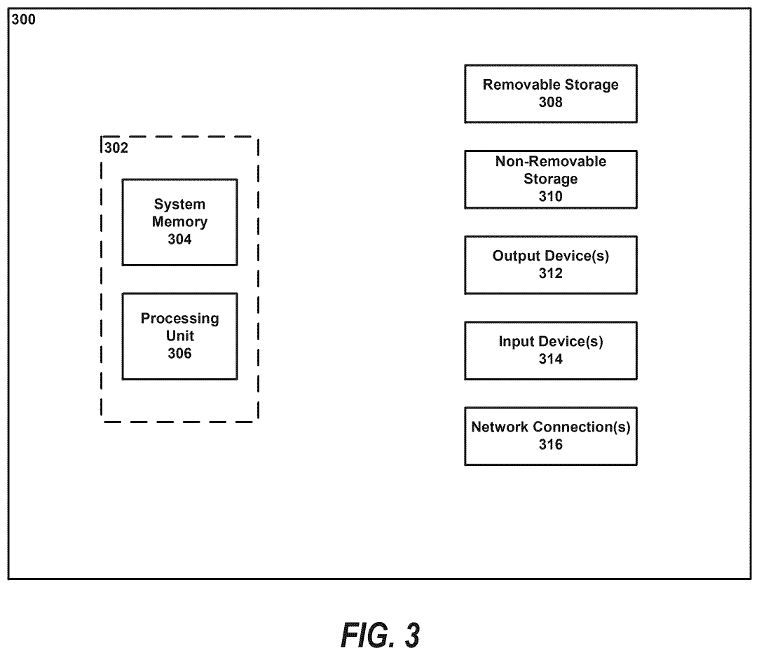

The controller 108 can include any or all of the features of the computing device 300 described with reference to FIG. 3. The controller 108 can be configured to control the system to perform the methods of soil measurement described with reference to FIGS. 2A and 2B. For example, the controller 108 can be configured to control and/or receive data from any or all of the soil moisture sensor 104, spectrometer 106, vacuum pump 112 and/or peristaltic pump 120. Additionally, the controller 108 can optionally include a data logger configured to store measurements in memory and/or transmit measurements (e.g., by a wired or wireless network).

The soil moisture sensor 104 can optionally be a volumetric water content sensor. The present disclosure contemplates that different types of soil moisture measurement can be performed by the system of FIG. 1A. For example, the system of FIG. 1A can optionally estimate a soil matric potential, for example using a Van Genuchten model. Any number of soil moisture sensor(s) 104 can be used, and the soil moisture sensor(s) can optionally be spaced to measure soil moisture at different locations within the suction lysimeter 102.

The system of FIG. 1A can be configured to collect soil moisture in different ways. For example, the system of FIG. 1A can be configured as a suction lysimeter where one or more suction cups 114 are coupled to a vacuum pump 112. For example, four suction cups 114 can be used, and optionally the suction cup(s) 114 can be disposed at different locations within the suction lysimeter 102. One or more sample bottle(s) 116 can optionally be disposed between the suction cup(s) 114 and the vacuum pump 112 to collect soil moisture samples to be analyzed by the spectrometer 106.

Alternatively or additionally, the system can include a peristaltic pump 120 that can pump samples from the sample bottles 116 to the spectrometer 106.

The spectrometer 106 can optionally be a nitrate spectrometer. Alternatively or additionally, the spectrometer 106 can be a be a UV-Vis spectrometer. In some implementations, the spectrometer 106 is configured to measure light between 200 and 750 nanometers.

In some implementations, the sample bottles 116, vacuum pump 112, and/or peristaltic pump 120 can be configured to vent the sample bottles 116 to the outside air. FIG. 1B illustrates an example system configured to vent one or more sample bottles (e.g., four bottles).

With reference to FIGS. 2A-2B, example methods are shown according to implementations of the present disclosure.

FIG. 2A illustrates an example method of nitrate (e.g., NO₃—N) measurement that can be implemented according to implementations of the present disclosure.

At step 210, the method can include initiating a soil moisture measurement. Optionally, the soil measurement can include a soil matric potential. An example soil matric potential can be computed by a Van Genuchten model, which can optionally be represented by the following equation:

$${S}_{e} = \frac{θ - {θ}_{r}}{{θ}_{s} - {θ}_{r}} = \frac{1}{{[ 1 + {( - α h )}^{n} ]}^{m}}$$

Example soil matric potentials that can be measured by the systems and methods of the present disclosure at step 210 are between −30 kPa and −1500 kPa.

At step 220, the method can include acquiring a soil porewater sample by applying a vacuum prior to application the force on the suction lysimeters based on the soil moisture measurement. Acquiring a soil porewater sample at step 220 can be triggered by detecting that the soil matric potential is at a certain level, threshold, and/or range (e.g., −30 kPa to −1500 kPa or any subset of those ranges).

At step 230, the method can include estimating a nitrate concentration of the soil porewater sample using a spectrometer. Optionally, a compensated spectrum can be estimated which can minimize the effects of Dissolved Organic Carbon on nitrate measurements.

Optionally, steps 220 and 230 can be repeated any number of times. For example, a second water sample can be collected, and an additional nitrate concentration can be measured for the second water sample. This can allow the system and methods of the present disclosure to continuously monitor the moisture content at a location (e.g., checking for a moisture threshold every second, minute, hour, day, or other time interval), and sample the nitrate concentration at the location each time the moisture content exceeds a certain threshold at the location.

### Example Spectrometer

An example spectrometer that can be used in implementations of the present disclosure includes a UV-Vis spectrometer with a broad spectrum of measurement (e.g., 200-750 nm). The spectrometer can be configured to identify a “fingerprint” in the spectrum, including identifying two absorption signals within the parameter of interest's associated wavelength. As non-limiting examples, the nitrate compensated spectrum ranges from 200 to ~250 nm and the total dissolved solids compensated spectrum ranges ~375-750 nm. The example spectrometer measures both nitrate (as NO₃—N or NO₃) and total dissolved solids (Formazin turbidity unit, FTU, and/or Nephelometric Turbidity unit, NTU, and/or total suspended solids, TSS). Optionally, the spectrometer can be a spectrometer that is not sensitive to other substances (e.g., such as organic carbon and total dissolved solids). Dissolved organic carbon is known as a primary interference in UV absorbance spectroscopy for nitrate analysis in natural samples, and implementations of the present disclosure can include systems without filters. Optionally, the systems and methods can include performing multi-point calibration on the spectrometer where reference measurements are input into the spectrometer's internal software, and an auto-calibration is performed. Optionally, the calibration performed is based on Principal Component Analysis (PCA) and Partial Least Squares Fit (PLS). This multi-point calibration can be performed once and may not require additional calibrations.

Example performance characteristics of a spectrometer that can be used in implementations of the present disclosure include: Nitrate measurement range: 0-100 (as NO₃—N); Total dissolved solids range: 0-460 (NTU/FTU); Operating temperature: 0-50 degrees Celsius; Fast measurement intervals, every 10 seconds (programmable); and/or network-enabled (e.g., “internet of things” and/or web server on board).

### Example Measurement Method

An example prototype was constructed with 4 suction probes and 2 soil moisture sensors. The system was configured to extract soil pore water from 2 locations. We had 2 suction probes and a single soil moisture sensor in each location. As described with reference to FIG. 1A, the number of soil pore water suction probes and soil moisture sensors can be increased or decreased. For example, the system can function if there's at least one probe and one soil moisture sensor.

The system and methods can use sensors that report soil moisture as a volumetric water content (VWC, %). VWC is the ratio of the volume of water to the unit volume of soil. This value is an indicator of how much water is in the soil but not how much of that water is available or can be extracted by the plant. Soil matric potential (SMP), also known as soil suction or soil water tension, represents the force at which water is being held in the soil (by water molecules binding onto soil particles and/or each other). A suction greater than a soil's SMP must be applied to extract water from the soil.

Implementations of the present disclosure can use soil volumetric water content sensors. Soil volumetric water content sensors can be cheaper and easier to install. However, unlike soil volumetric water content, which can vary significantly between different soil types, matric potential allows for a more consistent interpretation of soil water status across different soils. Therefore, the systems and methods described herein can convert soil volumetric water content to soil matric potential using the Van Genuchten (1980a) model:

$${S}_{e} = \frac{θ - {θ}_{r}}{{θ}_{s} - {θ}_{r}} = \frac{1}{{[ 1 + {( - α h )}^{n} ]}^{m}}$$

where Sₑ [−] is the effective saturation or relative water content, θ is the volumetric water content [cm³ cm⁻³], θₛ is the saturated volumetric water content [cm³ cm⁻³], θᵣ is the residual volumetric water content [cm³ cm⁻³], and α [L⁻¹], n [−], and m [−] are fitting parameters and h [L] is the soil matric potential.

### Example Study

A study was performed of an example implementation of the present disclosure, including an automated soil pore water sampling and nitrate detection system as described in FIG. 1A. The system is programmed to collect soil pore water sample(s) if specific conditions are met. After the sampling period has passed, the collected samples are individually directed to a nitrate-specific UV-Vis spectrometer for measurement. The novelty of this system is that sampling is event-based and therefore discontinuous. The aim of this approach is to avoid system failure due to environmental conditions that would naturally inhibit sample collection.

The example system automates a sampling method and pairs it with a nitrate-specific UV-Vis spectrometer for nitrate detection. The example system includes two soil moisture sensors, four ceramic suction cups, four sample collection bottles, four peristaltic pumps, a vacuum pump, tubing, and a nitrate-specific UV-Vis spectrometer. Again, the system is not limited to this specific number of units. It could be made to include different numbers and combinations of each part in various implementations.

The example system was configured to be deployed onto a field of interest. Soil moisture sensors and suction cups are configured to be installed into the soil at desired sampling depth(s).

Once deployed, the example system can be configured to operate with different sampling rates (e.g., how often soil moisture is checked) and different thresholds (e.g., what range of soil moisture measurements initiates a nitrate measurement). Optionally the sampling rates and/or thresholds are user configurable.

FIG. 2B summarizes an example flowchart showing operations that can be taken by the disclosed systems to perform nitrate sensing based on the detection of volumetric water potential and/or soil matric potential using soil moisture sensors. Example operations include:
(1) Measure and record soil moisture at regular ‘time’ intervals. (2) At a ‘time’ of day, soil moisture is checked. IF a soil moisture ‘threshold’ is NOT met, nothing happens aside from continuing to measure and record soil moisture at the ‘time’ interval. IF a soil moisture ‘threshold’ IS met, the following occurs:
(a) The vacuum is powered on and begins to maintain a vacuum (user specified) within the attached bottles for a ‘time’ duration. Note: each suction cup is paired to their own sample bottle which is where the extracted soil pore water is directed into. Additionally, the spectrometer is powered on and begins to measure for nitrate at a ‘set time’ interval.
(b) After some ‘time’, the vacuum is powered off.
(c) Suction/vacuum in the bottles is released and a valve is switched to open the bottles to the atmosphere.
(d) Peristaltic pumps are turned on one-at-a-time for some ‘time’. Note there is one pump per bottle and they operate in sequence. Note: this directs the solution collected in the bottles towards the spectrometer. At the same time the first pump is triggered on, the spectrometer is powered on and begins to measure at a ‘time’ interval.
(e) Once this cycle is complete, the spectrometer is turned off and soil moisture measurements continue as scheduled.

It should be understood that steps 1 and 2, and sub steps (a)-(e), can be repeated any number of times, and/or in different orders. Another non-limiting example implementation is shown in FIG. 2C.

The present disclosure further contemplates that measurements of nitrate concentrations can be transmitted to a remote computing device (e.g., a device including any of the features of the computing device 300 shown in FIG. 3). For example, the remote computing device can be configured to display nitrate concentrations, record nitrate concentrations over time, and/or cause a nitrate (e.g., a nitrate fertilizer) to be applied to a field based on the nitrate concentrations measured.

As used herein, the terms “about” or “approximately” when referring to a measurable value such as an amount, a percentage, and the like, is meant to encompass variations of ±20%, ±10%, ±5%, or ±1% from the measurable value.

It should be appreciated that the logical operations described herein with respect to the various figures may be implemented (1) as a sequence of computer-implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by box 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

An example implementation of the present disclosure was constructed and tested.

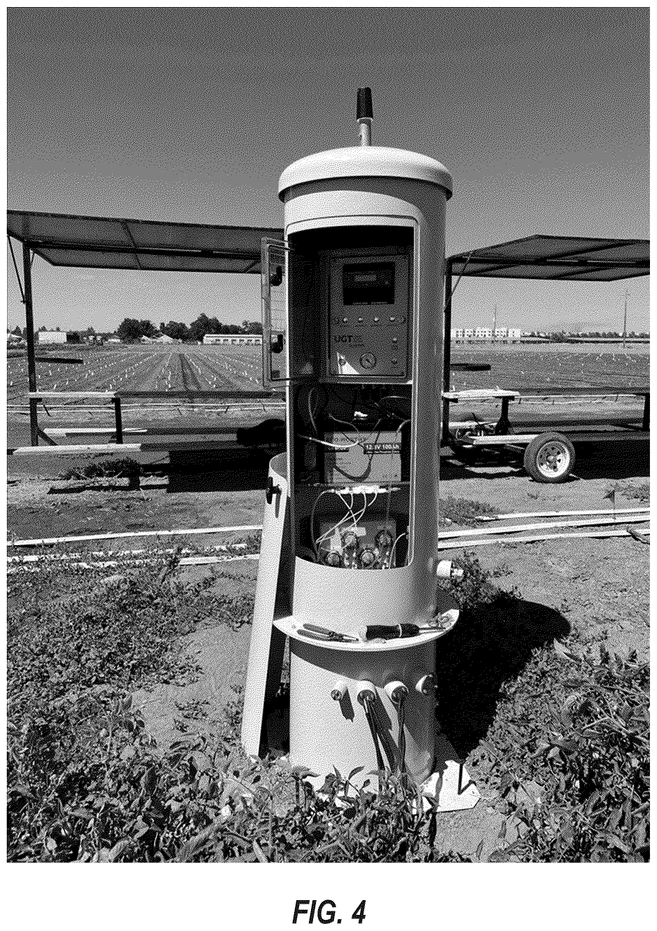

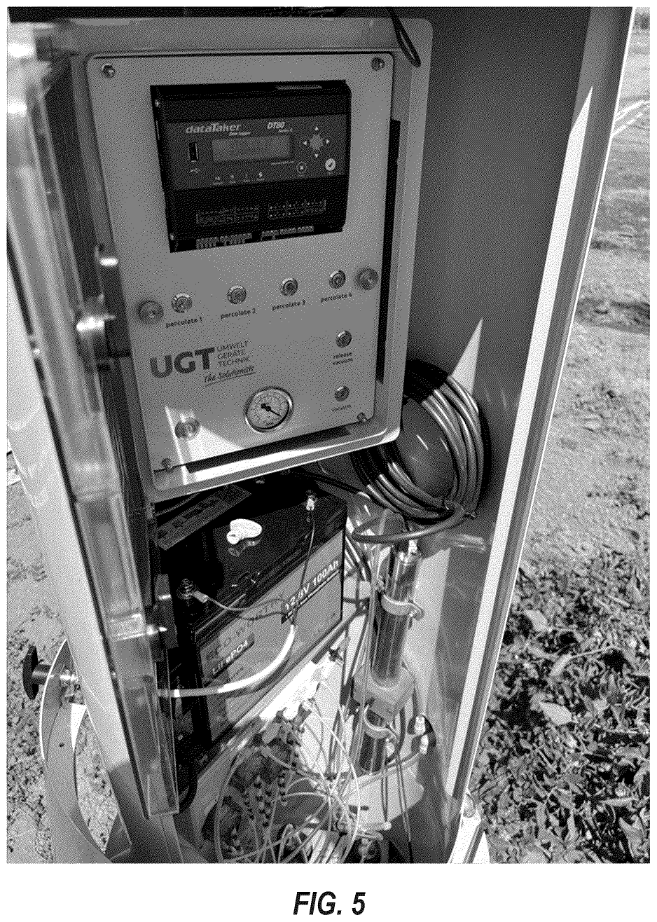

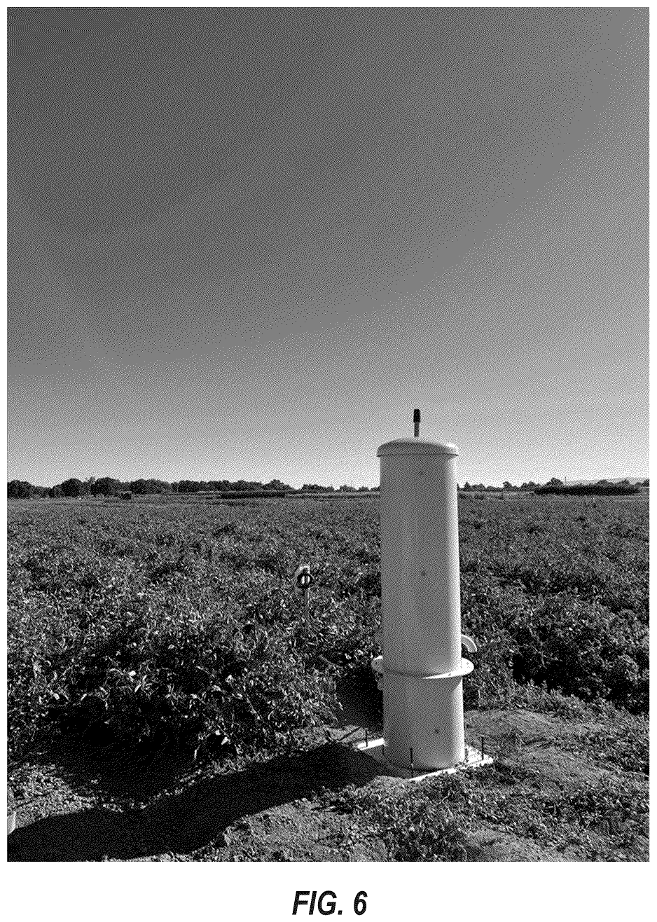

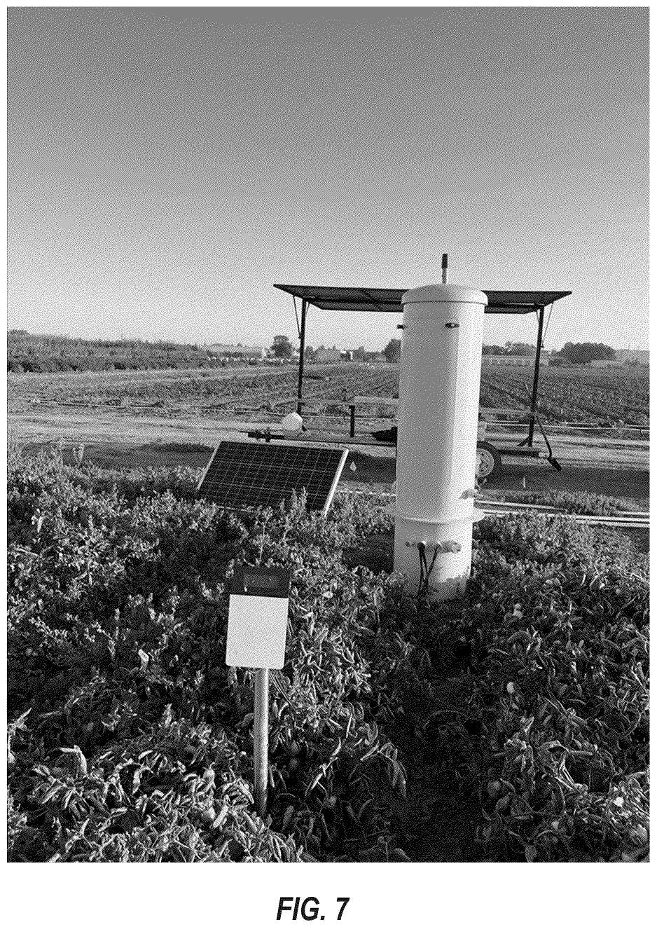

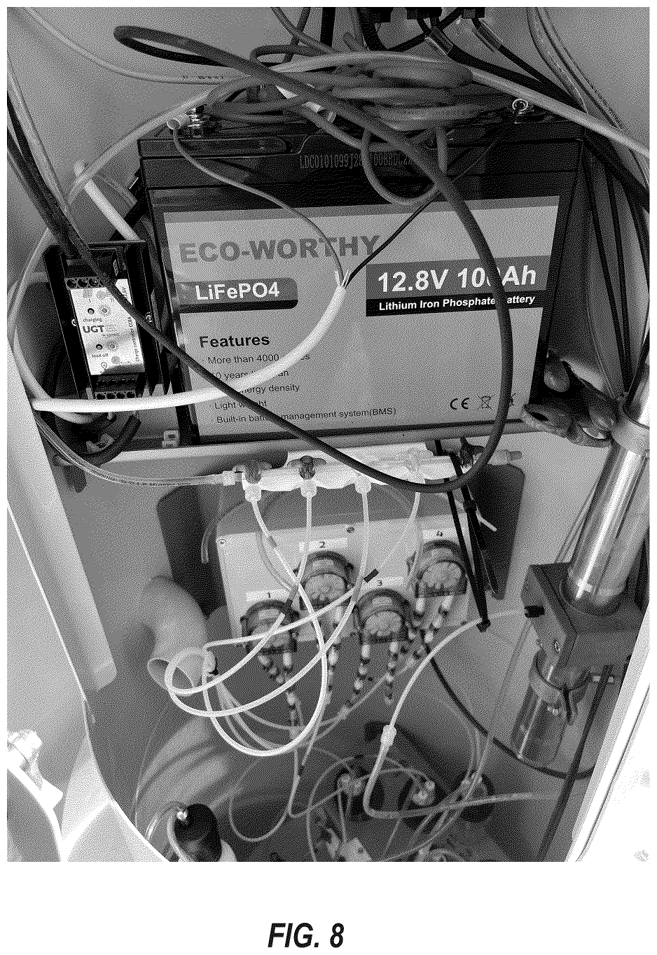

FIG. 4 illustrates an example implementation of the present disclosure deployed in a tomato field. FIG. 5 illustrates a perspective view of the interior of the example implementation shown in FIG. 4. FIG. 6 illustrates a rear-view of the example implementation of the present disclosure shown in FIG. 4. FIG. 7 illustrates a rear-view of the example implementation of the present disclosure shown in FIG. 4. FIG. 8 illustrates an interior view of the example implementation of the present disclosure shown in FIG. 4.

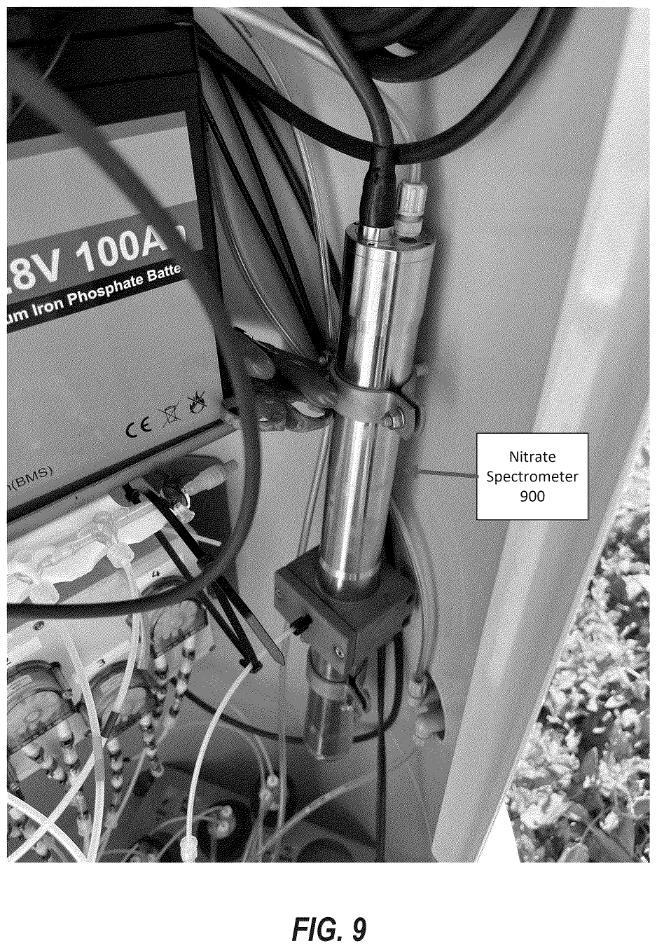

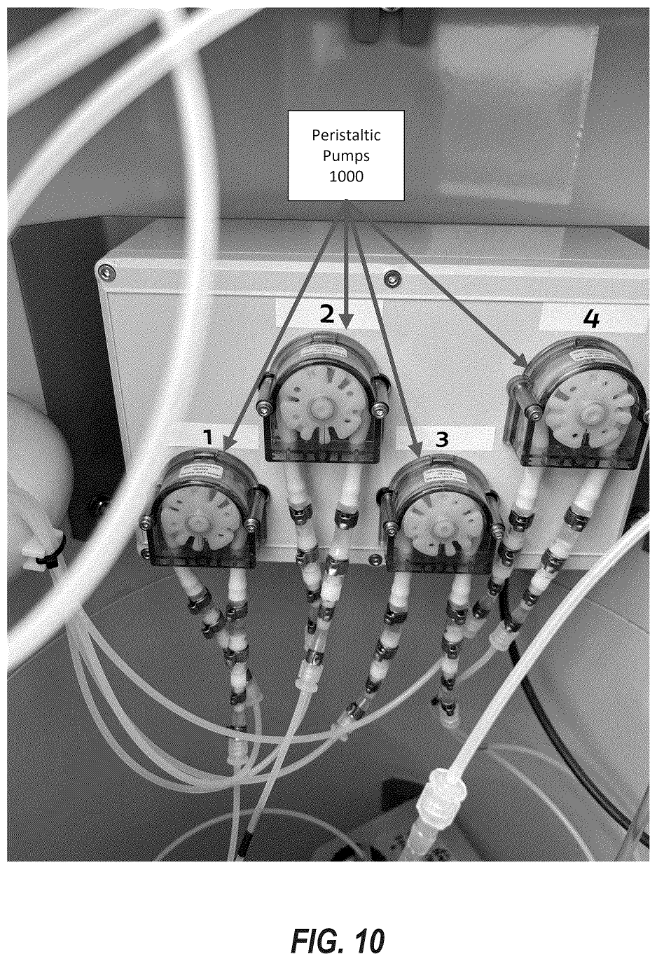

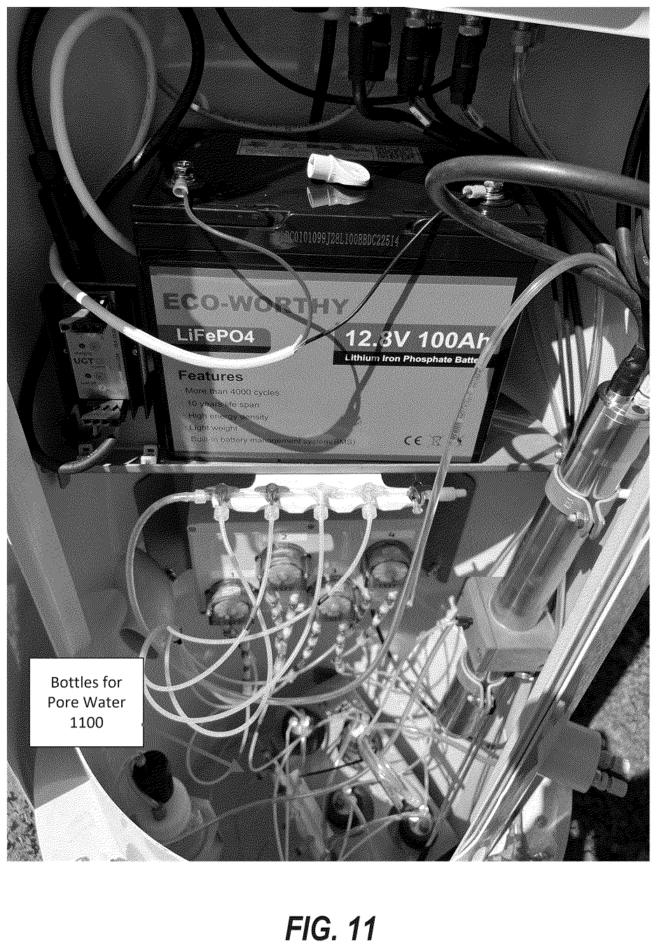

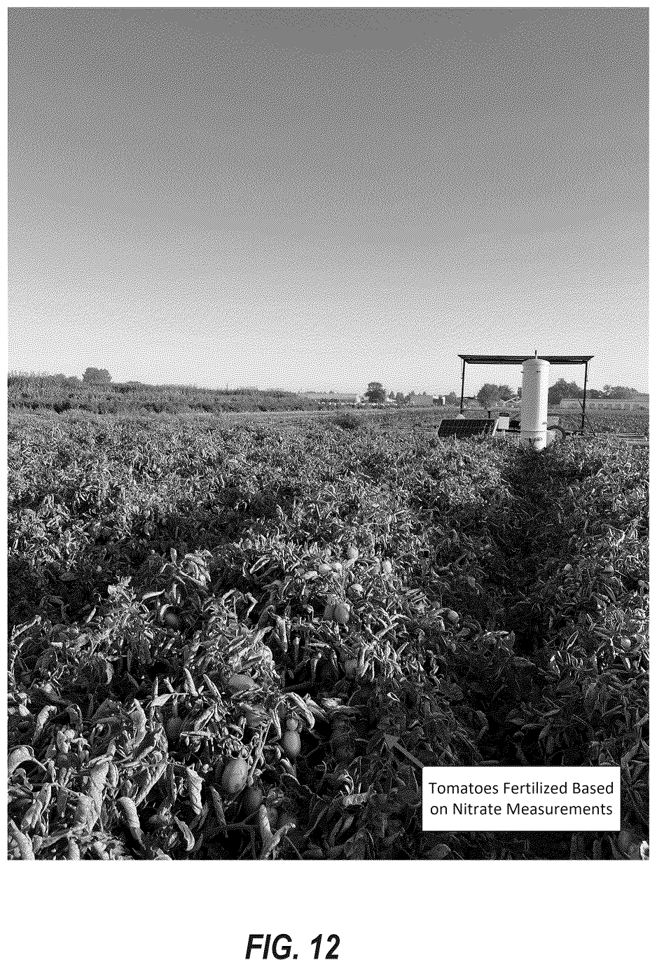

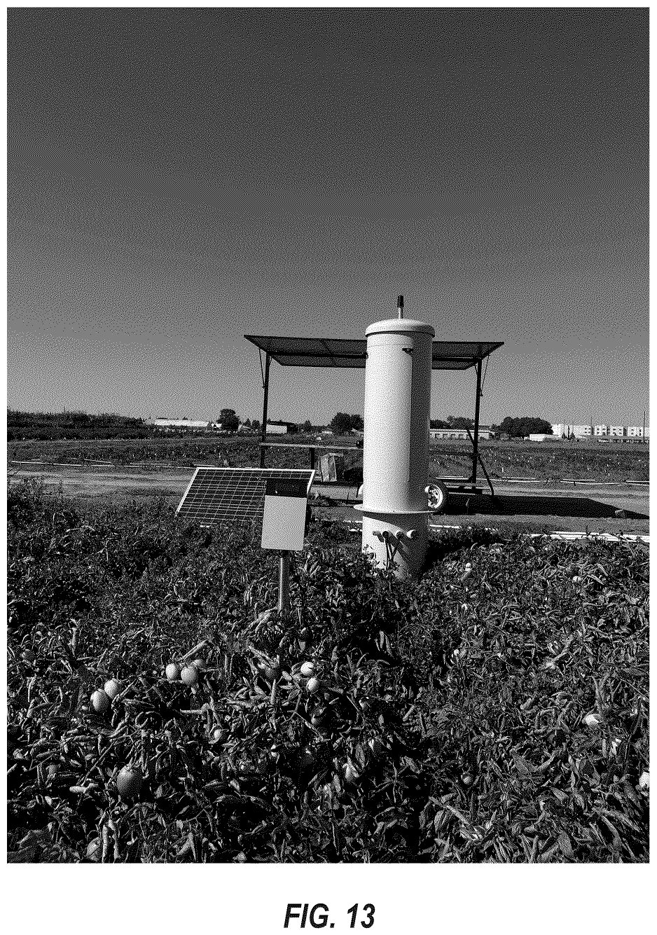

FIG. 9 illustrates an interior view of the example implementation of the present disclosure shown in FIG. 4, including an example nitrate spectrometer 900. FIG. 10 illustrates an interior view of the example implementation of the present disclosure shown in FIG. 4, including peristaltic pumps 1000. FIG. 11 illustrates an interior view of the example implementation of the present disclosure shown in FIG. 4 including bottles 1100 for pore water. In the example shown in FIG. 11, the bottles are glass bottles sold under the trademark “Duran” but it should be understood that any type of bottle can optionally be used. The suction lysimeter collects the pore water into the bottles 1100. Optionally, the number of bottles 1100 corresponds to a number of locations in the soil where suction lysimeters are used to collect pore water. Further, as described herein, the number of bottles 1100 can optionally correspond to the number of peristaltic pumps 1000, so that each peristaltic pump draw pore water from a respective bottle into the nitrate spectrometer 900 for measurement (e.g., sequential measurements of each bottle). FIG. 12 illustrates a rear-view of the example implementation of the present disclosure shown in FIG. 4 and a field of tomatoes fertilized based on nitrate measurements collected by the example implementation of the present disclosure. FIG. 13 illustrates a rear-view of the example implementation of the present disclosure shown in FIG. 4.

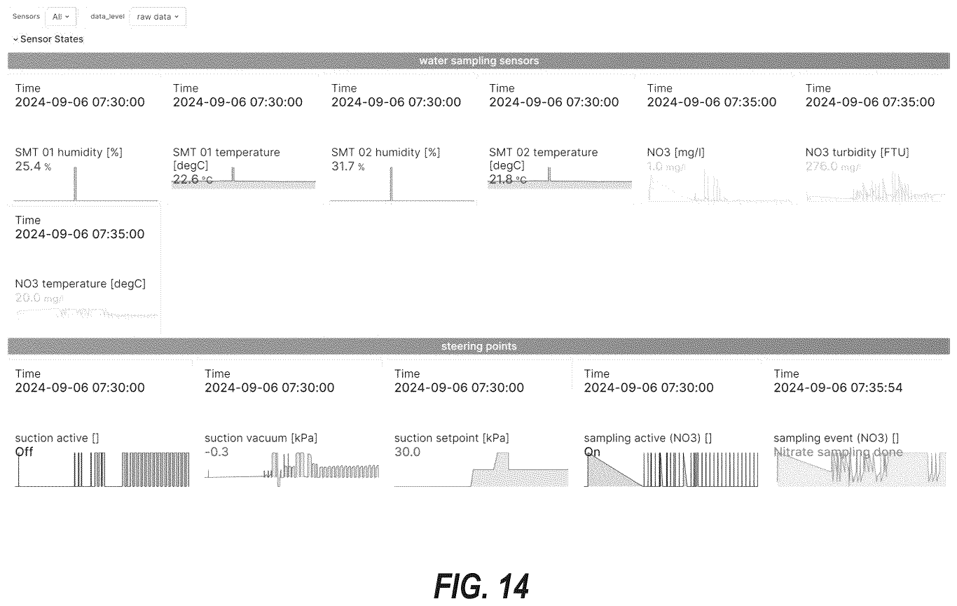

FIG. 14 illustrates an example user interface configured to output data collected from the example implementation shown in FIGS. 4-13, the example data shown represents an example deployment and testing period for the example implementation of the present disclosure.

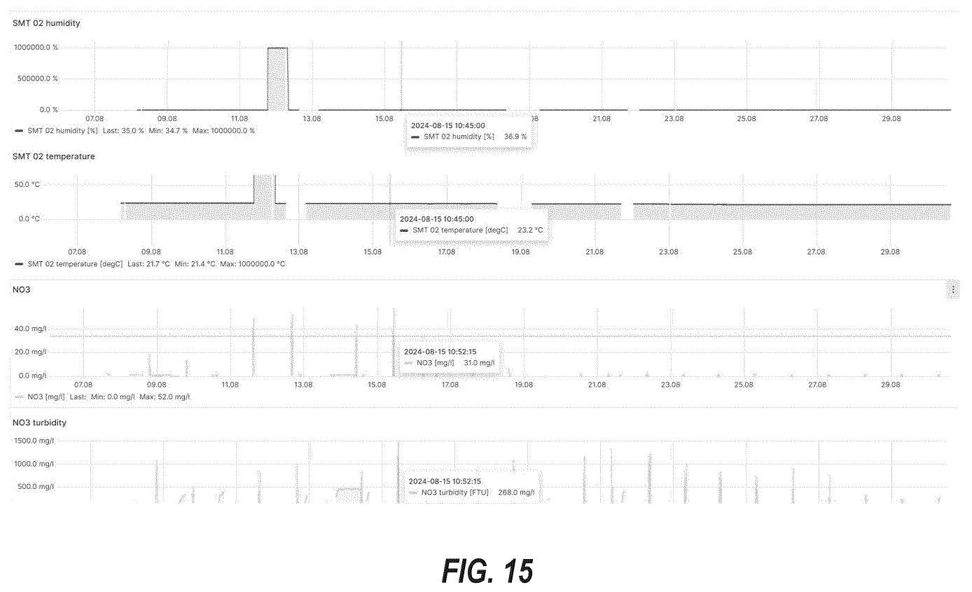

FIG. 15 illustrates an example user interface configured to display soil moisture (as SMT 02 humidity) and nitrate sensor data (as NO3), according to the example implementation shown in FIGS. 4-13.

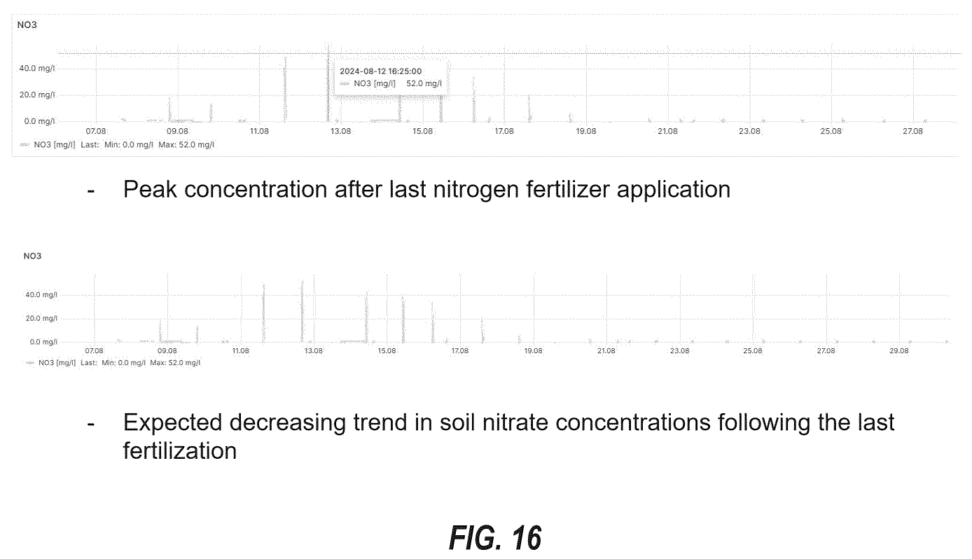

FIG. 16 illustrates example user interfaces configured to display soil nitrate concentrations, according to the example implementation shown in FIGS. 4-13.

FIG. 16: The last application of N fertilizer was on 8/6/2024. Soil nitrate concentrations peaked on 8/12/2024 (at 52 mg/L NO3-N). The system captured the expected fall in soil nitrate concentrations after this final application.

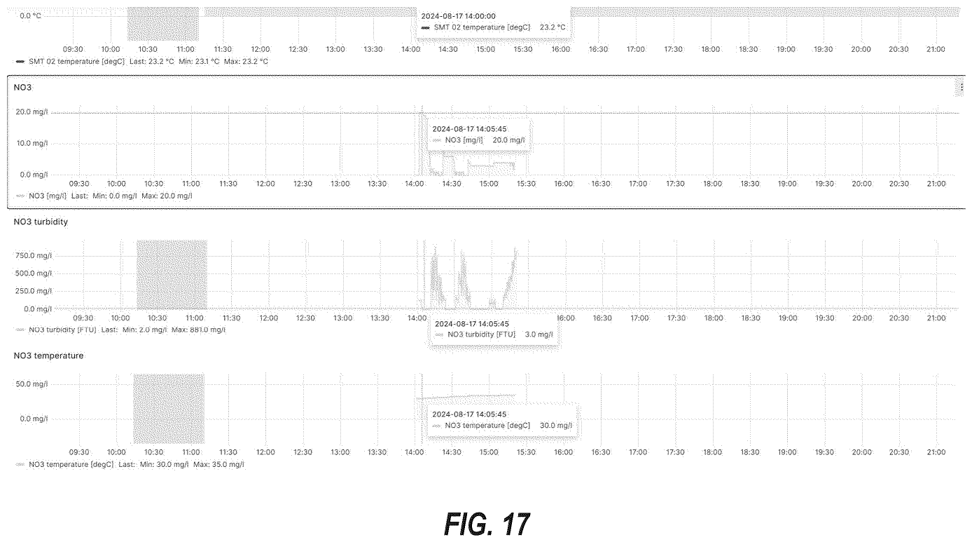

FIG. 17 illustrates a display of an example nitrate measurement event, according to an example implementation of the present disclosure.

The example system was equipped with two soil moisture sensors and four suction cups. When soil conditions are met there are four different locations within the soil where pore water is extracted from and measured for nitrate concentration. The example system can output the nitrate concentration data for display so that the user can see the different concentrations at the different locations by isolating the 4 peaks in nitrate concentrations. In this case, the first peak was around 20 mg/L. From the example installation documentation in the study, it is shown that this concentration is at a 45 cm depth.

### Example 2

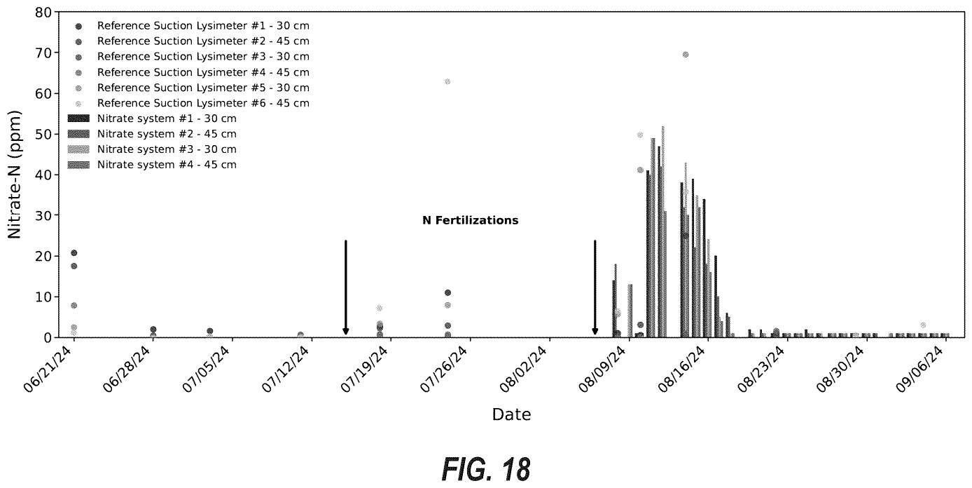

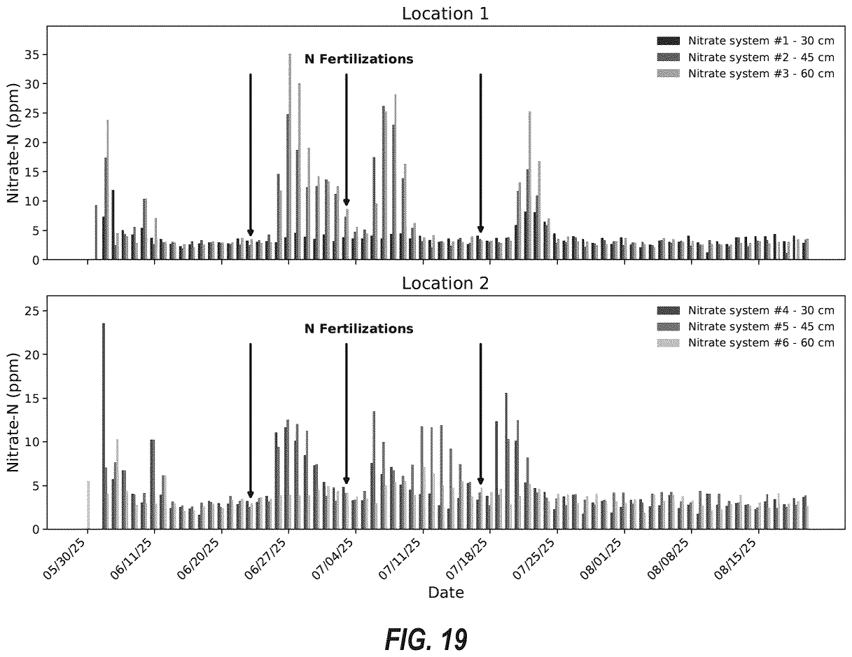

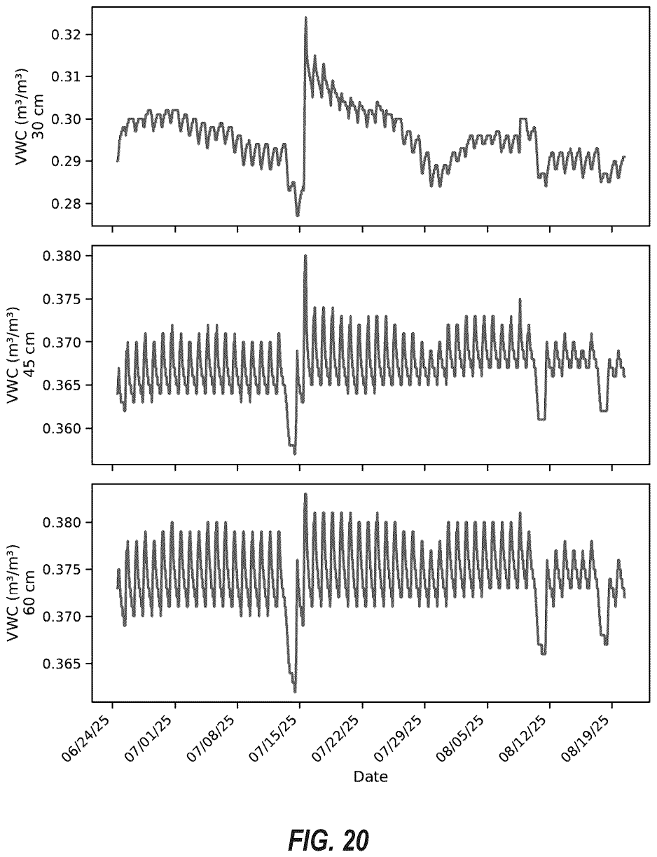

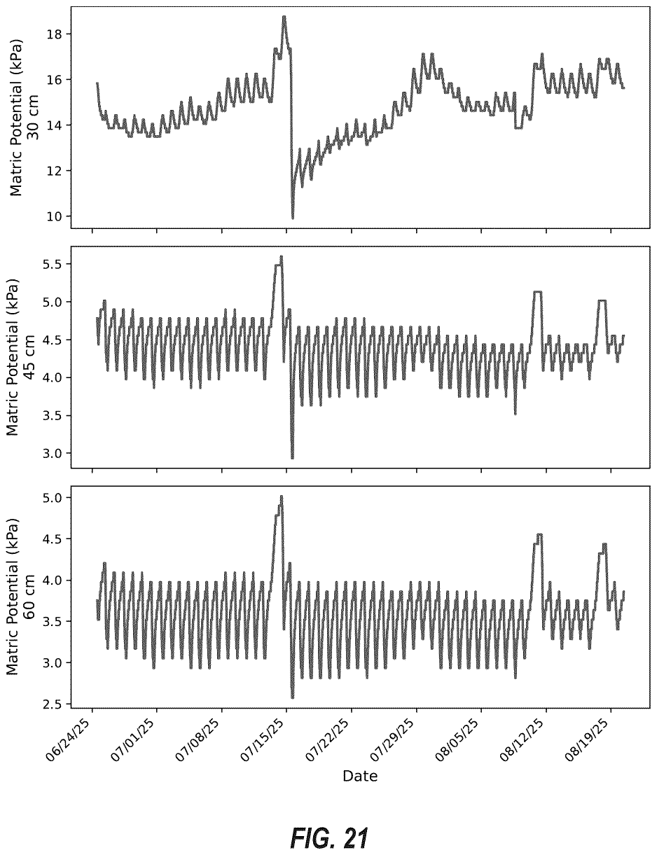

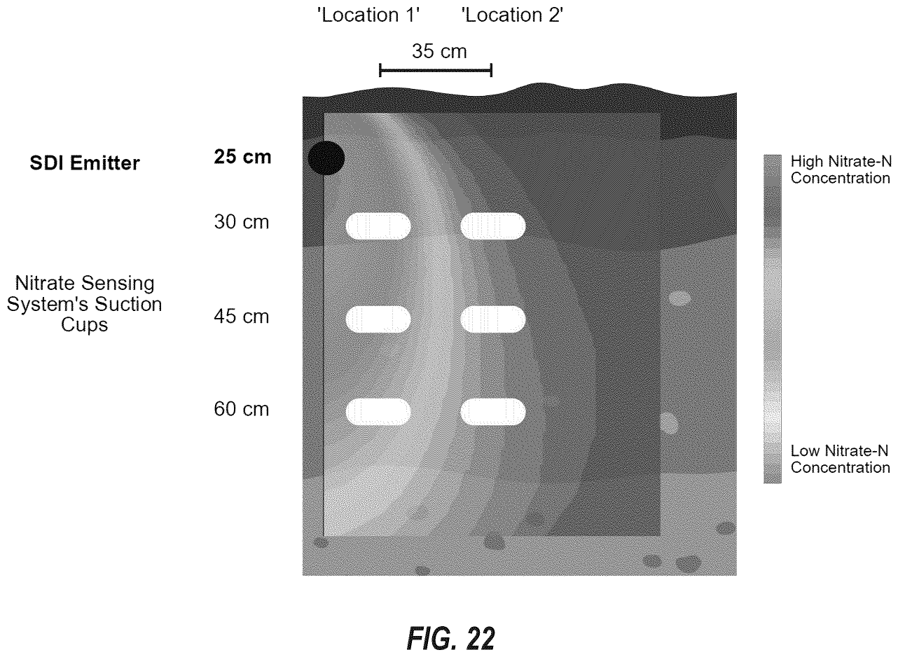

Data was collected in an example study to validate an example implementation of the present disclosure. Field trial data was collected at a farm processing tomatoes under subsurface drip irrigation (SDI) demonstrates the practical utility, robustness, and improvement of the proposed spectrometer-based real-time soil nitrate sensing system. The data show that the example implementation of a sensor can continuously quantify nitrate-N concentrations at multiple depths (e.g., 30, 45, and 60 cm) and lateral positions within the variably saturated zone surrounding an SDI emitter, capturing dynamic nitrate responses to discrete fertigation events and subsequent redistribution over time as shown in FIG. 19. When interpreted alongside coincident soil water status measurements as shown in FIG. 20's illustration of volumetric water content and FIG. 21's illustration of matric potential (derived from VWC using the Van Genuchten model as described herein). The example implementation enables direct identification of nitrate accumulation versus downward transport, providing early detection of conditions associated with nitrate leaching below the active root zone. Comparative results from the previous year further demonstrate strong agreement between the proposed sensor and conventional suction lysimeters, while offering substantially higher temporal resolution and eliminating the need for manual sampling as shown in FIG. 18. The deployment geometry illustrated in the system layout highlights the ability to resolve spatial nitrate gradients unique to SDI wetting patterns, which are not observable using existing point-based or episodic sampling approaches, as shown in FIG. 22. The data shown in FIGS. 18-22 further illustrates how the example implementation provides growers with actionable, real-time insight into fertigation efficiency and leaching risk, enabling reductions in fertilizer inputs and associated costs while addressing regulatory and environmental concerns related to groundwater nitrate contamination capabilities not available in current soil nitrate monitoring technologies.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A soil nitrate sensing system comprising:
a suction lysimeter;
a soil moisture sensor;
a spectrometer; and
a controller, wherein the controller is configured to:
estimate a soil moisture measurement by the soil moisture sensor;
based on the soil moisture measurement, acquire a water sample by the suction lysimeter; and
estimate nitrate concentrations as NO₃—N in the water sample using the spectrometer.

2. The soil nitrate sensing system of claim 1, wherein the soil moisture sensor comprises a volumetric water content sensor.

3. The soil nitrate sensing system of claim 2, wherein estimating a soil moisture measurement comprises estimating a soil matric potential.

4. The soil nitrate sensing system of claim 3, wherein the soil matric potential is computed by a Van Genuchten model.

5. The soil nitrate sensing system of claim 1, wherein the suction lysimeter comprises at least four suction probes.

6. The soil nitrate sensing system of claim 1, wherein the suction lysimeter is configured to sample water from at least two locations.

7. The soil nitrate sensing system of claim 1, wherein the soil moisture sensor comprises at least two sensor probes.

8. The soil nitrate sensing system of claim 1, wherein the soil moisture measurement comprises soil moisture at two locations.

9. The soil nitrate sensing system of claim 1, wherein the spectrometer comprises a UV-Vis spectrometer.

10. The soil nitrate sensing system of claim 1, wherein the spectrometer is configured to measure light between 200 and 750 nanometers.

11. The soil nitrate sensing system of claim 1, wherein estimating a nitrate (NO₃—N) concentration comprises estimating a compensated spectrum.

12. The soil nitrate sensing system of claim 1, wherein the soil lysimeter comprises a vacuum pump, a peristaltic pump, and a plurality of bottles configured in the controller to store a plurality of soil porewater samples based on sampling sequence.

13. The soil nitrate sensing system of claim 12, wherein the soil lysimeter is configured to vent the plurality of bottles.

14. A computer-implemented method of nitrate (NO₃—N) measurement comprising:
(A) initiating a soil moisture measurement;
(B) acquiring a soil porewater sample by applying a vacuum prior to application of a force on a suction lysimeter based on the soil moisture measurement; and
(C) estimating a nitrate concentration of the soil porewater sample using a spectrometer.

15. The computer-implemented method of claim 14, wherein estimating a soil moisture measurement comprises estimating a soil matric potential.

16. The computer-implemented method of claim 15, wherein the soil matric potential is between −30 kPa and −1500 kPa.

17. The computer-implemented method of claim 15, wherein the soil matric potential is computed by a Van Genuchten model.

18. The computer-implemented method of claim 17, wherein the Van Genuchten model comprises:
$${S}_{e} = \frac{θ - {θ}_{r}}{{θ}_{s} - {θ}_{r}} = \frac{1}{{[ 1 + {( - α h )}^{n} ]}^{m}}$$

19. The computer-implemented method of claim 14, wherein estimating a nitrate concentration of the soil porewater sample using a spectrometer comprises estimating a compensated spectrum which minimizes effects of Dissolved Organic Carbon on nitrate measurements.

20. The computer-implemented method of claim 14, further comprising repeating steps B and C to acquire a second water sample and estimate an additional nitrate concentration of the second water sample.

21. The computer-implemented method of claim 14, further comprising transmitting the nitrate concentration to a remote computing device.

22. The computer-implemented method of claim 14, further comprising determining an amount of nitrate to apply to a field based on the nitrate concentration.

23. The computer-implemented method of claim 14, further comprising applying a nitrate fertilizer to a field based on the nitrate concentration.
